(19) United States
(12) Patent Application Publication
(10) Pub. No.: US 2009/0036801 A1
(43) Pub. Date: Feb. 5, 2009

(54) Hand-eye coordination test instrument

(76) Inventors: Yu-Che Chuang, Tainan County (TW); Chiou-Jong Chen, Taipei City (TW); Yu-Pei Huang, Tainan City (TW); I-Hsing Huang, Rende Shiang (TW)

Correspondence Address: Frenkel & Associates, P.C., Suite 330, 3975 University Drive, Fairfax, VA, 22030, US

(21) Appl. No.: 11/888,605

(22) Filed: Aug. 1, 2007

(51) Int. Cl. A61B 5/103

(52) U.S. Cl. 600/595

(57) ABSTRACT

A hand-eye coordination test instrument includes at least a control device located in the test instrument, a LED stimulus array located on the surface of the test instrument and an input device. An operator can transmit test signals to the LED stimulus array through the control device. A testee receives reactions and sends them back through the input device to the test instrument. Through the control device the operator can judge and evaluate the hand-eye coordination condition of the testee.

### FIELD OF THE INVENTION

The present invention relates to a hand-eye coordination test instrument that transmits test signals through visual stimulus to a testee and capture and analyze related data to make a composite judgment regard hand-eye coordination capability of the testee.

### BACKGROUND OF THE INVENTION

Eyes, hands and the like are important body organs to perceive external environmental signals (such as light, colors on the surface of goods, smoothness of the surface of goods, and the like). The received signals are transmitted through sensory nerves to the brain to do synthesis, analysis and judgment. Then the brain issues commands to order bones and muscles through motor nerves to generate reactions.

If people's brains suffer from damages or were underdeveloped, the signals transferred from the eyes or other organs cannot be effectively and accurately judged. As a result the judgment of the brain is not accurate and the body cannot effectively receive the external signals. To people who are underdeveloped congenitally, for a long time there is no test instrument to provide accurate test for the people with such hindrance. The lacking of accurate judgment could result in delay of medical treatment.

### SUMMARY OF THE INVENTION

The present invention aims to provide a hand-eye coordination test instrument that can rapidly test the hand-eye coordination condition of a testee to promote to follow on accurate therapy, and also can help determination whether the testee suffers from mental or muscular fatigue.

To achieve the foregoing object the hand-eye coordination test instrument of the invention includes at least a control device, a LED stimulus array and an input device. The LED stimulus array is controlled through the control device by a desired test procedure set up by an operator, and the LED stimulus array transmits signals to a testee. The testee makes judgment and sends the commands through the input device feedback to the test instrument. Through the operator and the control device the hand-eye coordination condition of the testee can be evaluated by microprocessor.

By means of the hand-eye coordination test instrument the following functions can be achieved:

1. Instant test: Operator can control random lighting positions of LED lamps of the LED stimulus array through the control device and activate a system timer. The testee can transmit related commands through a handheld touch pen to the test instrument. Through a program module resided in a micro-processor system of the control device and the operator, the response time and the distance between the coordinate which the touch pen ticks and the coordinate of a LED lamp being lighted can be determined. Thereby the hand-eye coordination capability of the testeee can be obtained instantly without going through many other tests such as tests through medicines and the like.

2. Simple operation: Through the program module resided in the micro-processor system of the control device the random lighting position of the LED lamps can be controlled. The testee transmits the related signals through the handheld touch pen to the test instrument. Thus the hand-eye coordination capability of the testee can be determined. Operation is simpler and easier.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of an embodiment of the invention in a use condition.

FIG. 2 is a schematic process flow of the invention.

FIG. 3 is a flow chart of the invention.

FIG. 4 is a perspective view of another embodiment of the touch panel of the invention.

### DETAILED DESCRIPTION OF THE PREFERRED EMBODIMENTS

The hand-eye coordination test instrument of the invention aims to test hand-eye coordination condition of a testee through reactions of vision and visual receiving. The visual sense is one of the tools for human body to receive ambient signals. The visual sense transfers external signals to the brain which makes judgment and transmits commands to other organs such as hands, feet and the like to generate coordination and reactions.

In the event that problems occur to the coordination and reaction, it usually takes a great deal of test procedures and medicines to determine the coordination and reaction condition of a testee. The present invention provides a novel test method in which the eyes receive signals and the brain makes judgments. Then the commands are transferred by hand to do activities related to coordination and reaction. Through the test instrument and the operator, the hand-eye coordination capability of the testee can be determined.

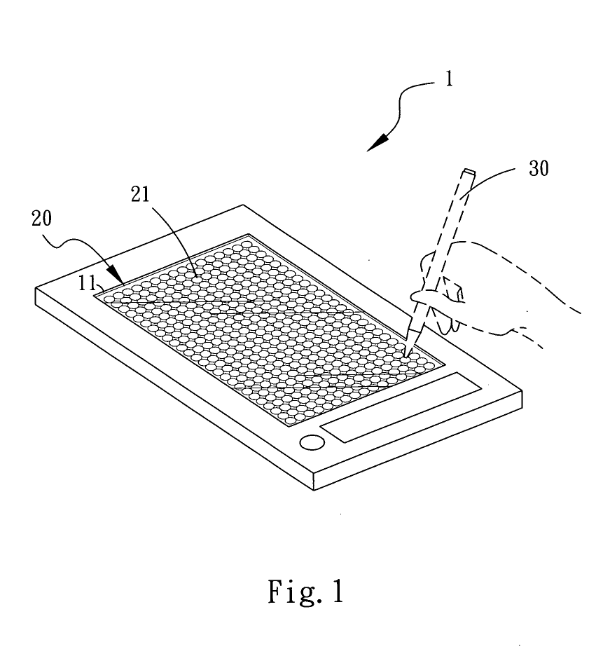

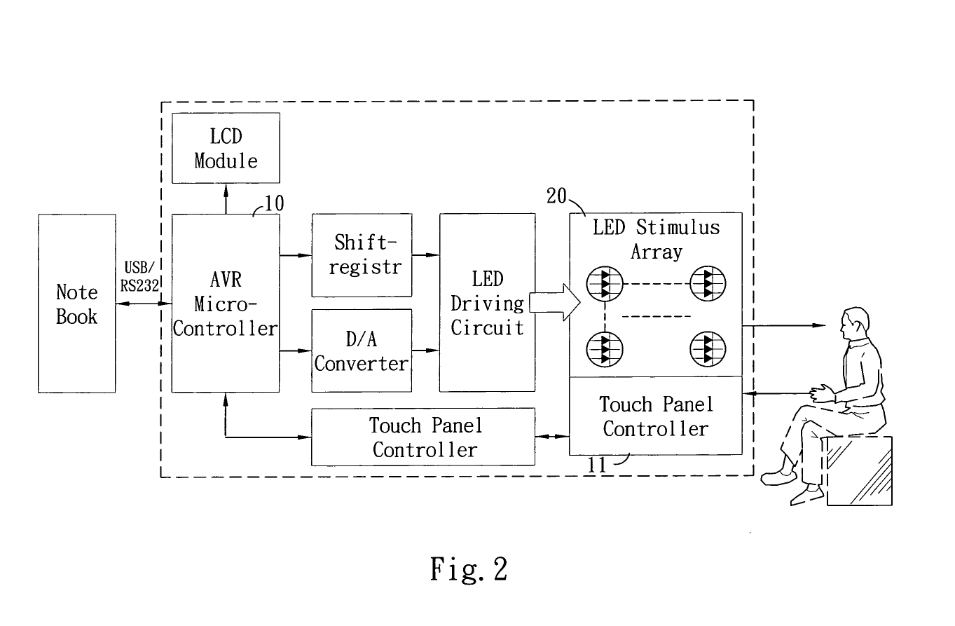

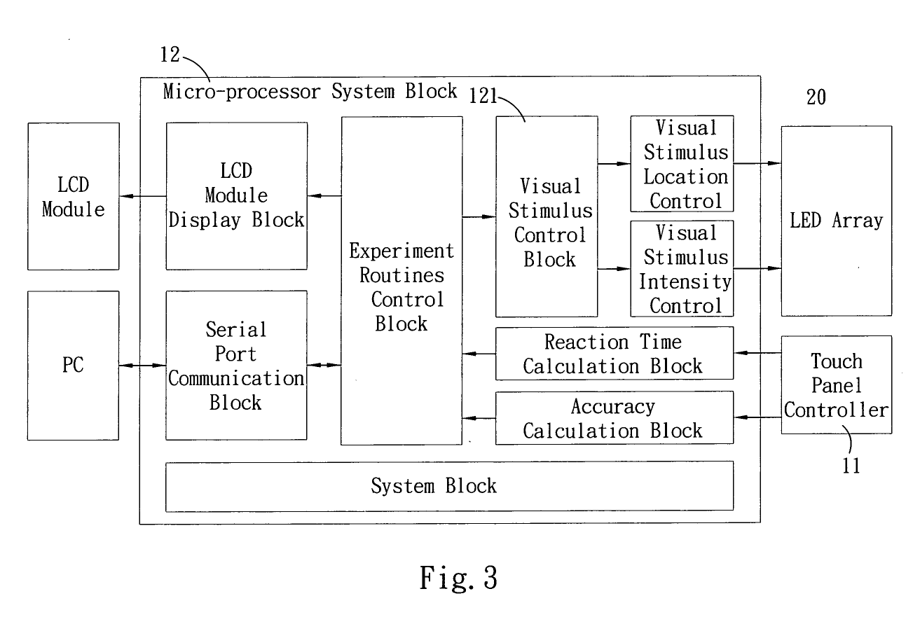

Refer to FIGS. 1 and 2 for an embodiment of the hand-eye coordination test instrument 1 of the invention. It includes at least a control device 10, an LED stimulus array 20 and an input device 30. The control device 10 includes at least a touch panel controller 11 and a micro-processor system 12. The micro-processor system 12 contains a test program module. Related command signals are input through the touch panel controller 11 and judged by the program module in the micro-processor system 12. Then the LED stimulus array 20 generates test-related reactions by lighting the lamps. The signals entered in the scope of the touch panel controller 11 are determined by the program module of the micro-processor system 12 as shown in FIG. 3.

The LED stimulus array 20 includes a plurality of LED lamps 21 and are controlled by a visual stimulation control unit 121 of the micro-processor system 12. Through the touch panel controller 11 the related command signals are entered. The program module of the micro-processor system 12 makes judgment and orders the LED stimulus array 20 to generate test-related reactions, such as program setting of lighting sequence of the LED lamps 21, lighting duration of the LED lamps 21 and the like. The LED stimulus array 20 may be other output devices such as a touch screen, LCD, or the like.

The input device 30 receives the signals from the LED stimulus array 20 provided by the testeee and transfers the commands to the hand-eye coordination test instrument 1. Based on the signals the hand-eye coordination test instrument 1 and an operator can make judgments. The input device 30 may be a handheld ticking article such as a touch pen, light pen or the like.

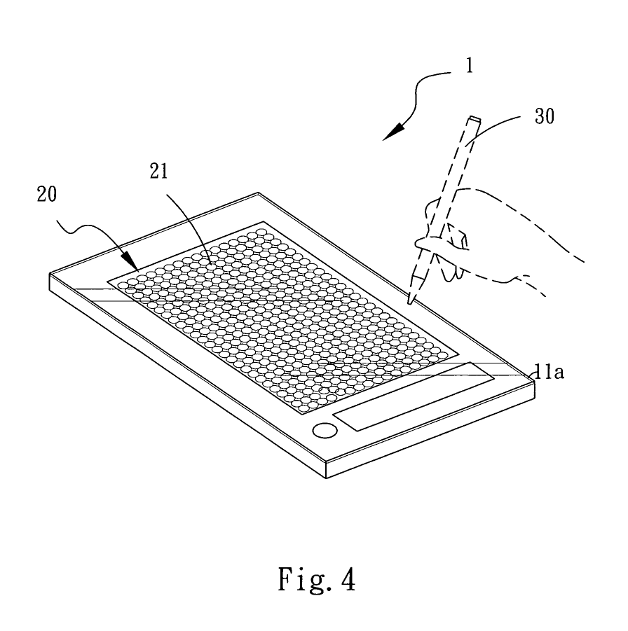

Referring to FIGS. 1, 2 and 3, the test-related procedures of the hand-eye coordination test instrument 1 are set up by the operator through the touch panel controller 11 of the control device 10. The touch panel controller 11 transmits the procedures to the control device 10 which has the program module resided therein to interpret the procedures set by the operator. The test procedures set by the operator are sent to the LED stimulus array 20. The LED lamps 21 of the LED stimulus array 20 can be lighted at different positions and duration. Referring to FIG. 4, the touch panel controller 11a can also cover an area larger than the displaying scope of the LED lamps 21. Hence even if the input location of the operator is far away from the lighting position of the LED lamp 21, the signal transmitted to the control device 10 from the touch panel controller 11 can be used to determine the distance difference between the input location of the operator and the lighting position of the LED lamp 21.

When the eyes of the testee receive the related visual signals of the lighting position, sequence and duration of the LED lamps 21 of the LED stimulus array 20 resulting from the test procedure set by the operator, the testee's brain will react to the signals. The testee holds the input device 30 by both hands. The testee's brain upon reaction to the test procedures will signal the testee's hands. The signals which are sent from the testee's brain to the testee's hands can be sent back to the LED lamps 21. The program module in the micro-processor system 12 can judge and synthesize the reaction signals of the testee to determine the hand-eye coordination condition of the testee, then transmits to the touch panel controller 11. The operator synthesizes the judged data provided by the hand-eye coordination test instrument 1 to get the test result of the hand-eye coordination condition of the testee.

The control device 10 of the hand-eye coordination test instrument 1 may also be linked to a computer which has a processing module to implement the related procedures of the invention.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A hand-eye coordination test instrument, comprising at least:
a control device to control an LED stimulus array and receive signals of an input device;
an LED stimulus array to transmit signals of the control device; and
an input device to transmit signals of a testee to the control device.

2. The hand-eye coordination test instrument of claim 1, wherein the control device includes a touch panel controller located on the surface of the hand-eye coordination test instrument and a micro-processor system located in the hand-eye coordination test instrument.

3. The hand-eye coordination test instrument of claim 2, wherein the touch panel controller covers an area larger than the LED stimulus array.

4. The hand-eye coordination test instrument of claim 2, wherein signals entered in the area covered by the touch panel controller are judged by a program module resided in the micro-processor system.

5. The hand-eye coordination test instrument of claim 2, wherein the micro-processor system has a program module resided therein.

6. The hand-eye coordination test instrument of claim 5, wherein the program module controls setting of desired test programs in the LED stimulus array.

7. The hand-eye coordination test instrument of claim 1, wherein the LED stimulus array is located on the surface of the hand-eye coordination test instrument and includes a plurality of LED lamps.

8. The hand-eye coordination test instrument of claim 1, wherein the LED stimulus array is a touch screen.

9. The hand-eye coordination test instrument of claim 1, wherein the LED stimulus array is a LCD.

10. The hand-eye coordination test instrument of claim 1, wherein the input device is a touch pen or a light pen.

11. The hand-eye coordination test instrument of claim 1, wherein the control device is linked to a computer which has a processing module to perform functions same as the hand-eye coordination test instrument.
